(19) United States
(12) Patent Application Publication
(10) Pub. No.: US 2011/0218459 A1
(43) Pub. Date: Sep. 8, 2011

(54) MEDICAL EXAMINATION AND/OR TREATMENT DEVICE

(76) Inventors: Paul Beasley, Oxfordshire (GB); Oliver Heid, Erlangen (DE); Timothy Hughes, Erlangen (DE)

(21) Appl. No.: 13/028,967

(22) Filed: Feb. 16, 2011

(30) Foreign Application Priority Data
Mar. 4, 2010 (DE) 10 2010 010 191.5

(51) Int. Cl. A61B 5/11

(52) U.S. Cl. 600/595

(57) ABSTRACT

A medical examination and/or treatment device has an apparatus for detecting the movement of at least one part of the body of an examination subject that changes its space coordinates periodically or aperiodically, wherein the apparatus includes at least one transmitter (8) transmitting the FMCW signal and at least one receiver (11) for receiving the FMCW signal reflected by the moving part of the body and is embodied for determining (3) the movement-induced changes in the space coordinates of the part of the body from the frequency shift of an FMCW signal reflected by the part of the body.

### CROSS-REFERENCE TO RELATED APPLICATIONS

This application claims priority to DE Patent Application No. 10 2010 010 191.5 filed Mar. 4, 2010. The contents of which is incorporated herein by reference in its entirety.

### TECHNICAL FIELD

The invention relates to a medical examination and/or treatment device having an apparatus for detecting the movement of at least one part of the body of an examination subject that changes its space coordinates periodically or aperiodically.

### BACKGROUND

The movement of a part of a body of a patient frequently presents difficulties within the framework of a medical examination and/or treatment, because e.g. problems due to motion artifacts can arise as a result of the cyclically changing position or size of an organ in space. An action known as triggering, specifically the emitting of an activation signal (trigger signal) at regular intervals in order to initiate, for example, the acquisition of an image, is used in order to perform an examination consistently at an identical time instant in a movement cycle. Different approaches are disclosed for reducing such motion artifacts through the use of a trigger signal or, as the case may be, for determining a movement of a part of the body, i.e. for physiological monitoring. These include, for example, ECG for recording cardiac movement, special chest belts having stress force stretch strips for detecting respiratory movement or magnetic-resonance-based systems.

It is known from U.S. Pat. No. 4,513,748 to determine the heart rate of a patient through the use of a Doppler signal, wherein two RF signals of different frequencies are directed toward the patient whose heart is to be examined. A frequency-shifted, high-frequency Doppler signal reflected by the patient provides information about movements of the patient's respiratory system. At the same time a frequency-shifted, low-frequency Doppler signal reflected by the patient provides information relating to both respiratory and cardiac movements. The values of the Doppler components associated with the heartbeat are comparatively weak in this case and are amplified by means of an amplifier such that ultimately the motion of the heart can be satisfactorily sensed and measured.

### SUMMARY

According to various embodiments, an improved medical examination and/or treatment device can be provided having a particularly precise detection of a physiologically induced movement of a part of a patient's body.

According to an embodiment, a medical examination and/or treatment device having an apparatus for detecting the movement of at least one part of the body of an examination subject that changes its space coordinates periodically or aperiodically, wherein the apparatus includes at least one transmitter transmitting the FMCW signal and at least one receiver for receiving the FMCW signal reflected by the moving part of the body and is embodied for determining the movement-induced changes in the space coordinates of the part of the body from the frequency shift of an FMCW signal reflected by the part of the body.

According to a further embodiment, the transmitted FMCW signal may have a predetermined frequency or a predetermined frequency range. According to a further embodiment, the transmitted FMCW signal can be variable on the transmitter side. According to a further embodiment, the apparatus may include a filter for filtering aperiodic changes in the determined space coordinates. According to a further embodiment, the transmitter and the receiver can be arranged directly on the medical examination and/or treatment device and/or on mounting devices contained in a treatment room and/or on stands that are freely positionable in the treatment room. According to a further embodiment, the transmitter and/or the receiver can be embodied as an array.

According to another embodiment, in a method for detecting the movement of at least one part of the body of an examination subject that changes its space coordinates periodically or aperiodically by means of an apparatus for detecting the movement of the part of the body, in particular using a medical examination and/or treatment device as described above, wherein the method comprises: the apparatus transmits an FMCW signal via at least one transmitter, which FMCW signal, after being reflected by the moving part of the body, is received by at least one receiver, and determines the movement-induced changes in the space coordinates of the part of the body from the frequency shift between transmitted and received FMCW signal.

According to a further embodiment of the method, the transmitter may transmit an FMCW signal of a predetermined frequency or a predetermined frequency range. According to a further embodiment of the method, a filter associated with the apparatus may filter aperiodic changes in the determined space coordinates. According to a further embodiment of the method, the apparatus may determine a respiratory or cardiac movement from the frequency shift as a function of the frequency or frequency range transmitted by the transmitter.

### BRIEF DESCRIPTION OF THE DRAWINGS

Further advantages, features and details of the invention will emerge from the below-described exemplary embodiment and with reference to the drawings, in which:

FIG. 1 shows a schematic drawing of a medical examination and/or treatment device in a first embodiment variant,

FIG. 2 shows a schematic drawing of a medical examination and/or treatment device in a second embodiment variant, and

FIG. 3 shows a schematic drawing of a medical examination and/or treatment device in a third embodiment variant.

### DETAILED DESCRIPTION

According to various embodiments, a medical examination and/or treatment device of the type cited in the introduction, may comprise at least one transmitter transmitting the FMCW signal and at least one receiver for receiving the FMCW signal reflected by the moving part of the body and which is embodied for determining the changing space coordinates due to the movement of the part of the body from the frequency shift of an FMCW signal reflected by the part of the body.

According to various embodiments, physiological movement can be detected from motion-induced changes in the space coordinates of a part of the body. According to various embodiment, the space coordinates are determined by means of an FMCW signal.

In the case of FMCW signals, the frequency shift (phase relationship) between transmit and receive signal can be evaluated. This is accomplished by the transmission of a time-continuous, frequency-modulated signal of defined length, said signal being very long in relation to the signal round-trip time. The signal round-trip time is the time between transmission of the FMCW signal by the transmitter and reception of the FMCW signal by the receiver; it is dependent on the propagation speed of the signal.

According to various embodiments, the FMCW signal is reflected as a frequency-shifted signal by a part of the body of an examination subject. That is to say that the FMCW signal radiated onto the part of the body has a different frequency from the FMCW signal reflected therefrom. In this case the frequency shift of the FMCW signal provides information about the space coordinates of the irradiated part of the body. What is meant by this, however, are not the absolute space coordinates, but relative space coordinates resulting from the proportionality between the frequency shift of the FMCW signal and the distance of the irradiated object from the object transmitting or receiving the FMCW signals. The distance is therefore a measure for the relative space coordinates of the part of the body and logically dependent on them. The relative space coordinates of the part of the body can therefore be deduced indirectly from the frequency shift of the FMCW signal. To that extent a time-varying frequency shift is always based on a movement of a part of the body which expresses itself in the change in its space coordinates.

The space coordinates can change aperiodically or periodically according to the movement. Periodic changes in the space coordinates in particular allow deductions to be made about periodic changes in the movements of one or more parts of the body, due, say, to the regular contraction of the heart according to the pulse rate.

As a result of the movement of a part of the body changing its space coordinates, which movement is detected by the apparatus, the latter can if necessary generate a trigger signal (activation signal) which controls the medical examination and/or treatment of the examination subject. Accordingly, a medical examination and/or treatment can be performed as a function of the trigger or activation signal consistently at identical time instants referred to an e.g. periodically moving part of the body.

The apparatus comprises at least one transmitter transmitting the FMCW signal and at least one receiver for receiving the FMCW signal reflected by the moving part of the body. Accordingly, the frequency shift is produced from the difference between the frequency of the FMCW signal transmitted by the transmitter and that of the signal received by the receiver. In this case transmitter and receiver are directed toward the part of the body that is to be examined or treated, which can also include a region of the body. It is of course possible for a plurality of transmitters and receivers to be assigned to the apparatus. Particularly advantageously, each transmitter can be assigned a specific receiver so that if there are a plurality of transmitters it is always possible to identify unequivocally which receiver receives FMCW signals from which transmitter.

According to further embodiments, it is provided that the transmitted FMCW signal has a predetermined frequency or a predetermined frequency range. It may therefore be that within the scope of the aforementioned assignment of transmitters and receivers said assignment is effected by way of corresponding predetermined frequencies or frequency ranges. A transmitter then communicates with a receiver exclusively or predominantly via a frequency specific to them or, as the case may be, via a corresponding frequency range. Interference with other transmitter-receiver pairs is precluded because these communicate with one another on different frequencies or, as the case may be, in different frequency ranges, i.e. by means of other FMCW signals.

Furthermore it can be advantageously possible by means of the setting or variation of the frequency transmitted by a transmitter or of a frequency range of a transmitted FMCW signal to emphasize the space coordinates and hence the movement of particular parts of the body. High frequencies of an FMCW signal in the range of 100 GHz or more do not penetrate deeply into the human body, so such FMCW signals mainly contain information relating to the respiratory cycle. FMCW signals at frequencies around 1 GHz, on the other hand, yield information relating to both the respiratory cycle and the cardiac cycle. According to various embodiments, it is therefore possible to detect movements of different parts of the body such as e.g. the lungs or the heart even with just one transmitter and one receiver.

In addition there are physiological processes which run predominantly aperiodically and consequently supply interfering, aperiodic frequency-shifted FMCW signals (cf. the peristalsis of esophagus or intestine). For this reason it is beneficial if the apparatus includes a filter for filtering aperiodic changes in the determined space coordinates. Aperiodic movements of the body which are reflected in aperiodic frequency shifts between transmitted and received FMCW signal can be detected and subsequently filtered so that they cause no falsification of the periodic FMCW signals. In this way incorrect deductions based on aperiodic movements of a part of the body in relation to periodic movements are precluded.

With regard to the mounting of the transmitter and receiver it is possible for these to be arranged preferably directly on the medical examination and/or treatment device and/or on mounting devices contained in a treatment room and/or on stands that are freely positionable in the treatment room. As a result thereof the transmitter(s) and receiver(s) can be arranged at many places within a treatment room and consequently, depending on the given space available, can be mounted or are mounted directly on the medical examination and/or treatment device by way of suitable fixing means. A second possible alternative where appropriate is the mounting on mounting devices which are contained in an examination or treatment room and which can be provided e.g. on the ceiling or walls. A third possibility according to various embodiments is the arrangement on freely movable stands, thereby resulting in a high degree of flexibility in the arrangement of one or more transmitters or receivers. The stands can be positioned as desired, possibly at predetermined positions, and simply moved away following completion of the examination or treatment. Obviously, in the case of a plurality of transmitters and receivers, an arrangement of a plurality of transmitters or receivers on one of the cited possibilities is also conceivable. Similarly it may be that e.g. a transmitter fixed to the ceiling communicates with a stand-mounted receiver assigned to it, or vice versa, consequently, in other words, that transmitters and receivers mounted on different objects communicate with one another.

Advantageously, the transmitters and/or receivers can be embodied in the form of an array. In this way the transmitted FMCW signal can be amplified by means of overlaying (constructive interference). Equally, the sensitivity of a receiver to an FMCW signal is increased, for which reason an overall improvement in transmission and reception characteristics of transmitter and receiver respectively can be achieved.

Furthermore, according to various further embodiments, in a method for detecting the movement of at least one part of the body of an examination subject changing its space coordinates periodically or aperiodically by means of an apparatus for detecting the movement of the part of the body, in particular using a medical examination and/or treatment device having an apparatus according to the above description, the apparatus transmits an FMCW signal via at least one transmitter, which FMCW signal after reflecting off the moving part of the body is received by at least one receiver, and determines the movement-induced changes in the space coordinates of the part of the body from the frequency shift between transmitted and received FMCW signal.

The method according to various embodiments is executed in accordance with the steps described hereinbelow. After an examination subject such as a patient has been positioned e.g. on a patient examination table, a transmitter directed toward the examination subject or, more precisely, toward the part of the body that is to be examined or treated transmits an FMCW signal onto the region that is to be examined or treated, which FMCW signal is reflected by the examination subject and received by a correspondingly aligned receiver. In this case, due to the reflection, the FMCW signal received by the receiver has a different frequency or frequency range from the FMCW signal originally transmitted by the transmitter, resulting in a frequency shift of the FMCW signal. From this frequency shift the apparatus can detect the movement-induced changes in the space coordinates of the part of the body.

If the movement of the part of the body and consequently the change in its space coordinates is periodic, the apparatus can make deductions in relation to a periodically running physiological process of the human organism, such as e.g. the respiration or the heartbeat, when, for example, the thorax of a human being is irradiated.

For this reason a trigger signal can possibly be determined or derived from the periodic, i.e. cyclical, change in the space coordinates, which signal is suitable for initiating a medical examination or treatment consistently at an identical time instant within a cycle.

Preferably the transmitter may transmit FMCW signals at a predetermined frequency or in a predetermined frequency range. According to the predetermined frequency or frequency range, the FMCW signal transmitted by the transmitter is sensitive to different regions of the examination subject. As described above, high-frequency signals in the range of 100 GHz mainly contain information relating to the respiratory cycle. Comparatively low-frequency signals at a frequency or in a frequency range of 1 GHz penetrate more deeply into the human body and accordingly provide information both in respect of the respiratory cycle and in respect of the cardiac movement. Through the selection and setting of the FMCW signal to a predetermined frequency or frequency range it is therefore possible to obtain information about different parts of the body with just one transmitter and receiver.

Equally it is possible through the selection and setting of an FMCW signal of a predetermined frequency or of a predetermined frequency range to assign a receiver specifically to a transmitter, since according to the frequency or frequency range of the FMCW signal emitted by the transmitter a receiver can only be sensitive to a corresponding frequency or the corresponding frequency range. Transmitter and receiver accordingly communicate via a frequency or frequency range that is uniquely assignable to them. This can be particularly advantageous in the case of a plurality of transmitters and/or receivers, since it is precisely in such cases that problems can occur in the assignment of transmitters and receivers.

Preferably a filter associated with the apparatus may filter aperiodic changes of the determined space coordinates. Aperiodic changes in the space coordinates being determined can reduce the significance of periodic changes in the determined space coordinates, since the frequency shifts underlying the aperiodic changes can have a negative effect in terms of the significance of periodic frequency shifts. The filter associated with the apparatus detects aperiodic changes and removes these from the spectrum of changes in the space coordinates detected by the apparatus, with the result that preferably only periodic changes in the determined space coordinates can be registered.

Preferably the apparatus may determine a respiratory or cardiac movement from the frequency shift as a function of the frequency or frequency range transmitted by the transmitter. As mentioned above, the frequency or frequency range of the FMCW signal transmitted by the transmitter is selectable, whereby the transmitter always transmits an FMCW signal of a predetermined frequency or predetermined frequency range. The frequency or frequency range of the FMCW signal can accordingly be set such that the FMCW signal is sensitive e.g. to a respiratory or cardiac movement. In this case correspondingly high frequencies yield mainly information in respect of the respiratory system, whereas lower frequencies in comparison therewith additionally furnish information in respect of the movement of the cardiac system.

As explained with reference to FIG. 1, a schematic drawing of a medical examination and/or treatment device 1 is depicted in a first embodiment variant. In this case the medical examination and/or treatment device 1 includes an apparatus 3, here assigned to a control device 2, for detecting the movement of a part of the body of an examination subject periodically or aperiodically changing its space coordinates, the examination subject in this case being the patient 4 lying on a patient couch 5. The medical examination and/or treatment device 1 is also assigned an X-ray apparatus 6 having an X-ray tube 7. An X-ray detector, although present, is not shown. Disposed on the X-ray apparatus 6 are a transmitter 8 transmitting an FMCW signal (arrow 9) and a receiver 11 receiving an FMCW signal reflected by the patient 4 as indicated by the arrows 10. In this arrangement transmitter 8 and receiver 11 can be preferably embodied as arrays and connected to the apparatus 3 via suitable communication means 14. Owing to the space coordinates of the patient 4 or, more specifically, of the region on the latter that is to be examined or treated (in the present case the thorax) it transpires that the FMCW signal transmitted by the transmitter 8 has a different frequency or a different frequency range from the FMCW signal received by the receiver 11. The space coordinates of the part of the body of the patient 4 that is to be examined or treated can be determined indirectly from the frequency shift of the FMCW signal by way of the apparatus 3.

Due to movement there is a change in the space coordinates of the part of the body, i.e. of the chest region in the present example, this being detected by the apparatus 3 as a function of a changing frequency shift of the FMCW signal. The apparatus 3 can therefore determine aperiodic or periodic processes within the body or, more specifically, the part of the body of the patient 4 from the changing frequency shift of the FMCW signal, based on the movement-induced change in the space coordinates of the part of the body of the patient 4.

The FMCW signal transmitted by the transmitter 8 has a predetermined frequency or a predetermined frequency range. In particular in cases in which a plurality of transmitters 8 and a plurality of receivers 11 are provided, a unique assignment of a transmitter 8 to a receiver 11 is thus possible by way of a frequency or frequency range specific to them in each case. The receiver 11 is to this end embodied in such a way that it can receive only FMCW signals in the frequencies or frequency ranges transmitted by the transmitter 8 assigned to it. In such a case other transmitters 8 and receivers 11 would communicate with one another via FMCW signals of other frequencies or frequency ranges.

The frequency of the FMCW signal transmitted by the transmitter 8 is variable on the transmitter side, as a result of which said signal can be sensitive to specific parts of the body of the patient 4. By means of a single transmitter 8 and a receiver 11 assigned thereto it is therefore possible, through selection of a suitable frequency or frequency range of the FMCW signal, to register movements of different parts of the body of the patient 4. High frequencies of the FMCW signal in the range of 100 GHz in this case provide information about the respiratory system of the patient 4, while comparatively low frequencies of the FMCW signal in the range of 1 GHz supply information about the cardiac system of the patient 4. By means of a corresponding variation of the transmitted FMCW signal or, more specifically, of its frequency or frequency range on the transmitter side the apparatus 3 is therefore able to register the movement of different organs.

The apparatus 3 includes a filter (not shown here in further detail) which detects and filters aperiodic changes in the space coordinates of a part of the body that have been determined by the apparatus 3. Aperiodic movements which express themselves as aperiodic frequency shifts of the FMCW signal can overlay periodic frequency shifts, which are often of particular interest from the medical perspective, and consequently diminish their significance. Preferably the filter associated with the apparatus 3 therefore may filter all aperiodic frequency shifts of the FMCW signal such that primarily only periodic frequency shifts are sensed and measured.

Periodic frequency shifts or changes in the space coordinates of a part of the body are of interest, for example, when the apparatus 3 is to generate a trigger signal (activation signal) on the basis of which the control device 2 controls the X-ray apparatus 6 in such a way that the latter always records images at an identical time instant within a periodically running movement cycle of a part of the body.

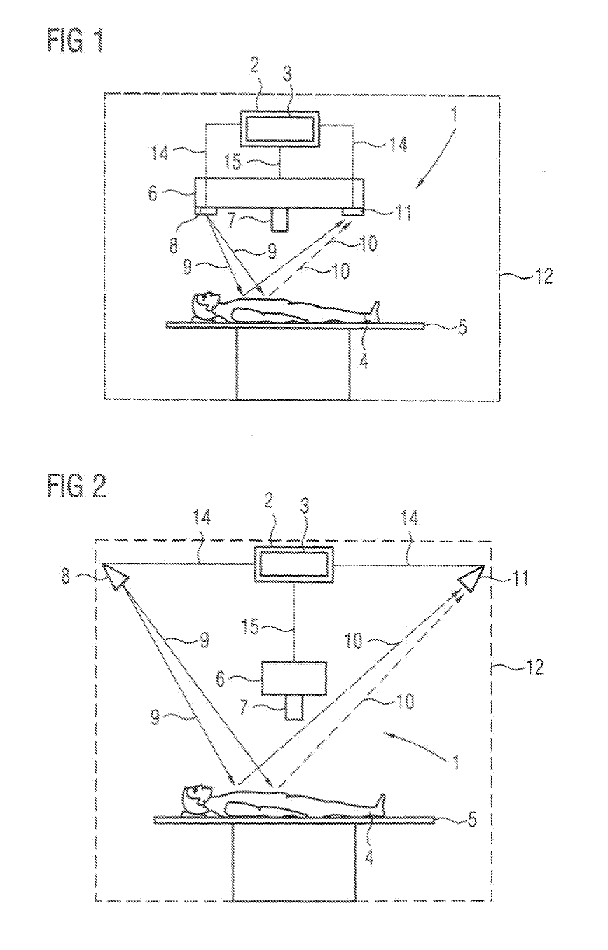

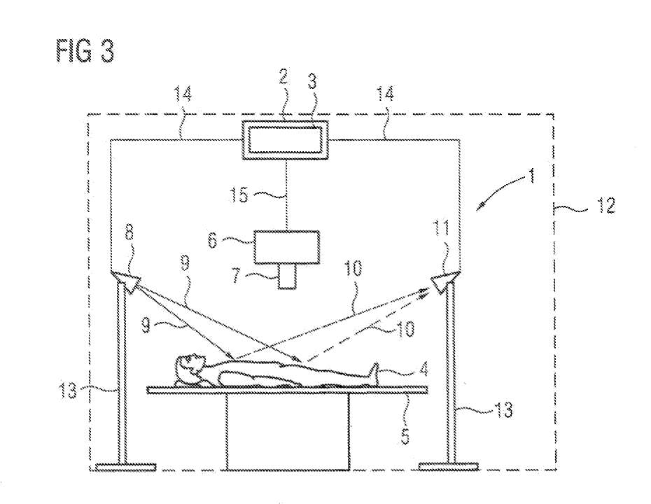

FIGS. 2 and 3 each show schematic diagrams illustrating a medical examination and/or treatment device according to further embodiment variants which differ from the embodiment variant according to FIG. 1 essentially by virtue of different arrangements of the transmitters 8 and receivers 11. According to FIG. 2 it is possible to install transmitter 8 and receiver 11 on the ceiling of a treatment room 12. This can be of advantage when it is not possible to mount the transmitter 8 and/or receiver 11 e.g. on the X-ray apparatus 6 due to space constraints. Affixing them to walls of the treatment room 12 is likewise conceivable.

According to FIG. 3 it is also possible to mount the transmitter 8 and/or the receiver 11 on stands 13 that can be moved in the treatment room 12. As a result the transmitter 8 and receiver 11 can be positioned virtually as desired, both in relation to the patient 4 and relative to each other. It may be that the stands 13 are not contained in the treatment room 12 prior to and after the medical examination or treatment, and are moved into the treatment room 12 and positioned there only during or shortly before the examination or treatment is carried out.

### LIST OF REFERENCE SIGNS

1 Examination and/or treatment device

2 Control device

3 Apparatus

4 Patient

5 Patient couch

6 X-ray apparatus

7 X-ray tube

8 Transmitter

9 Arrow

10 Arrow

11 Receiver

12 Treatment room

13 Stand

14 Communication means

What is claimed is:

1. A medical examination and/or treatment device having an apparatus for detecting the movement of at least one part of the body of an examination subject that changes its space coordinates periodically or aperiodically, comprising:
at least one transmitter transmitting the FMCW signal and
at least one receiver for receiving the FMCW signal reflected by the moving part of the body,
wherein the device is configured to determine the movement-induced changes in the space coordinates of the part of the body from the frequency shift of the FMCW signal reflected by the part of the body.

2. The medical device according to claim 1, wherein the transmitted FMCW signal has a predetermined frequency or a predetermined frequency range.

3. The medical device according to claim 1, wherein the transmitted FMCW signal is variable on the transmitter side.

4. The medical device according to claim 1, wherein the apparatus comprises a filter for filtering aperiodic changes in the determined space coordinates.

5. The medical device according to claim 1, wherein the transmitter and the receiver are arranged directly on the medical device.

6. The medical device according to claim 1, wherein the transmitter and the receiver are arranged directly on mounting devices contained in a treatment room.

7. The medical device according to claim 1, wherein the transmitter and the receiver are arranged directly on stands that are freely positionable in the treatment room.

8. The medical device according to claim 1, wherein the transmitter is embodied as an array.

9. The medical device according to claim 1, wherein the receiver is embodied as an array.

10. The medical device according to claim 1, wherein the transmitter and the receiver are embodied as an array.

11. A method for detecting the movement of at least one part of the body of an examination subject that changes its space coordinates periodically or aperiodically by means of an apparatus for detecting the movement of the part of the body, the method comprising:
transmiting an FMCW signal via at least one transmitter,
after being reflected by the moving part of the body, receiving the FMCW signal by at least one receiver, and
determining the movement-induced changes in the space coordinates of the part of the body from the frequency shift between transmitted and received FMCW signal.

12. The method according to claim 11, wherein the transmitter transmits an FMCW signal of a predetermined frequency or a predetermined frequency range.

13. The method according to claim 11, comprising filtering aperiodic changes in the determined space coordinates.

14. The method according to claim 11, comprising determining a respiratory or cardiac movement from the frequency shift as a function of the frequency or frequency range transmitted by the transmitter.

15. The method according to claim 11, wherein the transmitted FMCW signal has a predetermined frequency or a predetermined frequency range.

16. The method according to claim 11, wherein the transmitted FMCW signal is variable on a transmitter side.

17. The method according to claim 11, wherein the transmitter is embodied as an array.

18. The method according to claim 11, wherein the receiver is embodied as an array.

19. The method according to claim 11, wherein the transmitter and the receiver are embodied as an array.

20. The method according to claim 11, wherein the method is performed by a medical examination and/or treatment device comprising the at least one transmitter transmitting the FMCW signal and the at least one receiver for receiving the FMCW signal reflected by the moving part of the body.
